(19) United States
(12) Patent Application Publication
(10) Pub. No.: US 2021/0401528 A1
(43) Pub. Date: Dec. 30, 2021

(54) Canister Caps

(71) Applicant: Angela K. Wilson, Englewood, NJ (US)

(72) Inventor: Angela K. Wilson, Englewood, NJ (US)

(21) Appl. No.: 17/012,194

(22) Filed: Sep. 4, 2020

Related U.S. Application Data
(60) Provisional application No. 63/045,314, filed on Jun. 29, 2020.

(51) Int. Cl. A61B 46/10; A61B 46/00; A61L 31/06; A61L 31/16

(52) U.S. Cl. CPC A61B 46/10; A61B 46/40; A61B 2090/0807; A61L 31/16; A61L 31/06

(57) ABSTRACT

This present invention relates to a disposable sterilization indicator cap for use with a medical cannister or device. The disposable cap includes an indicator word or symbol visible on its exterior surface, and may be made from a relatively lightweight material. The disposable cap further comprises a repositionable closure for accommodating medical cannisters and devices of various sizes and shapes. The disposable cap prevents used medical canisters and other devices from being inadvertently used on multiple patients without having been properly sanitized, and eliminates the cross contamination that can occur when a dirty canister is inadvertently used.

### CROSS-REFERENCE TO RELATED APPLICATION

The present application claims priority to, and the benefit of, U.S. Provisional Application No. 63/045,314, which was filed on Jun. 29, 2020 and is incorporated herein by reference in its entirety.

### BACKGROUND

The present invention relates generally to the field of protective coverings for suction canisters to be used within the medical field. More specifically, the present invention relates to a suction canister covering that visually indicates that the canister is clean or sterilized, and which allows medical workers to quickly and easily determine the status of the canister between patients, thereby preventing the spread of unnecessary infections due to the use of contaminated canisters. Accordingly, the present specification makes specific reference thereto. Nonetheless, it is to be appreciated that aspects of the present invention are also equally applicable to other similar applications, devices and methods of manufacture.

By way of background, a suction canister is a temporary container used to collect and store secretions or fluids removed from a patient's body. By way of example, suction canisters are used to store the contents from respiratory secretions from the nasal, oral, or tracheotomy passages, as well as gastric contents during a nasal gastric lavage. Further, during operative surgery and other medical and biological procedures, the medical suction canisters may also be used to collect other fluids from the patient including, without limitation, blood, saline, and any other fluids which may accumulate or must be removed and contained during the procedure and disposed of after the procedure. Unfortunately, the bodily and other fluids contained within the medical canister may carry germs, and can be a source of transmission of various diseases to different patients if the status of the containers is not closely monitored or if they are used by multiple patients without being properly sanitized between uses.

More specifically, such medical canisters are typically reusable, but need to be cleaned, sterilized and/or sanitized before being re-used. If the medical canister is not fully sanitized and the contaminated or previously used canister is mistakenly used for another patient, the contaminated or unsanitary canister may result in the transmission of bacteria, viruses, microbes and other undesirable pathogens from the first patient to the second patient, thereby potentially worsening the second patient's health or medical condition and/or prolonging the period of time the second patient must spend hospitalized or receive treatment.

Accordingly, it is imperative that the sanitary condition of all such medical canisters be routinely monitored and clearly known to all parties involved. As a practice, medical workers and other healthcare professionals may keep the clean and dirty canisters on two separate tables, carts or shelves in order to differentiate between the status of any two canisters, and to ensure that a contaminated container does not cross-contaminate a sanitary container. However, such practices are not always followed or successful, and may result in a contaminated medical canister being mixed in with the clean or properly sanitized canister, which can in turn lead to the problems referenced above. Therefore, an efficient canister identification and distinguishing method is needed in the healthcare profession to overcome potential errors in selecting the wrong container or canister.

Other methods of differentiating sanitized medical canisters from those that have been used include creating a tag or label for the clean or used canister and pasting or adhering the tag or label on the canister so that other users are aware of the sanitized status of the canister. However, while in use the medical professional must remember to remove the tag and if he or she forgets to do so, the dirty or used medical canister may be inadvertently mistaken for a clean canister and used for multiple patients, thereby potentially causing cross contamination and the potential spread of diseases. Further, because such tags are temporary, they may fall off after a period of time, which again leads to confusion as to whether or not a particular medical canister is clean and ready for use.

Additionally, the existing methods for indicating the sanitized status of the medical suction canister require a huge effort in managing both the dirty and clean canisters. Separating clean suction canisters from dirty canisters, writing tags for sanitized status, pasting or adhering the tags on the suction canisters, and other similar methods require a lot of effort from the medical professionals, and add additional responsibility to an already taxing job. Stated differently, the existing methods are seen by some as inconvenient, time-consuming and may often lead to inadvertent use of dirty suction canisters for multiple patients.

Therefore, there exists a long felt need in the art for a quick and easy method for differentiating between a dirty or used medical canister and a clean or sterilized canister, and for a covering that easily conforms to or covers the medical canister, equipment, and other medical devices to be properly identified. Additionally, there is a long felt need in the art for a medical canister identification device which requires less effort and time to use, and that prevents a medical professional from inadvertently using a medical canister for multiple patients. Moreover, there is a long felt need in the art for a medical canister identification device that prevents cross contamination and the spread of disease, germs, viruses and the like. Finally, there is a long felt need in the art for a sanitized status indicator for a medical canister or other medical equipment, which is relatively inexpensive to manufacture and safe and easy to use.

The subject matter disclosed and claimed herein, in one embodiment thereof, comprises a medical canister sterilization indicator device that is comprised of a disposable cap for a medical device, such as a suction canister, with an indicating or status word CLEAN or STERILIZED, or similar words or symbols written, printed or imaged on the top surface of the disposable cap or otherwise visible on the top of the device. That is, the wording or symbol can be provided on the side opposite the top visible side and still be visible through the cover, such as with providing a transparent or translucent cover. The disposable canister cap may be made from a lightweight clear plastic material, or glassine, which may add to the recyclability of the device, and includes an elastic or draw string that allows the cover to wrap or seal around the canister. In use, the disposable cap is applied over the top of a newly cleaned/sanitized suction canister, thereby allowing medical workers to quickly and easily determine the status of the canister, and whether it has been cleaned or sterilized between patients. Further, the cap may be easily secured to the device to avoid it from becoming inadvertently dislodged, and is both disposable and recyclable.

In this manner, the disposable canister cap of the present invention accomplishes all of the forgoing objectives, and provides a relatively quick, easy and effective solution to the need to distinguish sterilized medical suction canisters from the used and dirty canisters for the convenience of the medical professional. The disposable canister cap of the present invention can be sized by the user to specifically accommodate different sizes of suction canisters and other medical equipment, such as surgical trays, monitors, lights, and other machines that may appear in an operating theater, examination room, patient room, or the like.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a disposable cover for a medical device, such as a suction canister, that is capable of being removably placed on and covers many different sizes and shapes of suction canisters, medical devices, other equipment, and the like. More specifically, the disposable cap of the present invention is made up of a relatively lightweight material, such as a lightweight medical grade plastic, glassine, or other readily useable material, and comprises a securing mechanism such as, but not limited to, a continuous rubber band, an elastic strip, a drawstring or other closure that wraps around the medical canister or device and cinches the disposable cap thereto in a secure fashion. The continuous rubber band, elastic or other closure defines the opening for the cap or cover, and extends along the entire circumference or perimeter of the bottom portion of the disposable cap. The disposable cap further comprises an indicating word or symbol, such as CLEAN or STERILIZED on its top surface to indicate the current cleanliness status of the medical canister or other medical equipment.

In a further embodiment of the present invention, a disposable cap for a medical device such as a suction canister is provided, and is capable of being placed on and covering a variety of different sizes and shapes of suction canisters, medical devices and the like. The cap has a channel defining an opening of the cap and extending along a portion of the circumference or perimeter of the disposable cap, wherein an elastic or other draw cord is positioned within the channel to slide and lock the cap to completely cover or encompass the medical device or suction canister to be identified. Additionally, the top portion forms a cavity adapted to receive the canister or medical device and, as above, has an indicator word or symbol such as CLEAN or STERILIZED on an outer top surface to indicate the current cleanliness status of the canister contained therein.

In yet another embodiment of the present invention, a method of constructing the canister cap of the present invention is described and involves the steps of first cutting a circular or other shaped piece of transparent or translucent material of a desired perimeter, depending on the ultimate size desired by the user or the maker of the medical device. Then a strip of elastic material, draw string, or other closure is cut which has an ultimate length less than the perimeter of transparent elastic plastic material. Then the strip of elastic or closure material is placed sufficiently inward of the outer perimeter of the circular or shaped piece of transparent or translucent material, so as to leave enough material to fold over and cover the closure strip entirely, and to be sewn or secured, such as by adhesive to the material.

In yet another embodiment of the present invention, a method of covering a medical container or device, such as a suction canister, to identify the cleaned and sterilized canisters to be used is disclosed. The method comprises the steps of initially providing a disposable plastic cover, and then placing a rubber band or elastic channel on the medical canister and stretching the band to cover the dimensions of the canister or device. The user then adjusts the fit of the disposable cover so that it completely encompasses or covers the canister or device, wherein the top portion of the cover contains indicia such as CLEAN or STERILIZED, or other symbology which is clearly visible to the healthcare professional. To use the suction canister or medical device, the plastic cover may be removed by a healthcare professional by stretching the elastic or rubber band and then removing the plastic cover from the medical device or canister. Further, the disposable cover may be discarded or recycled after its removal from the canister or medical device. The disposable plastic cover of the invention ensures that clean and sterilized suction canisters or medical devices are always available for a new patient as the suction canisters or medical devices remains clean under the plastic cover, and are easily identified with the above referenced identification mark or symbol. By using the plastic cover of the present invention, a dirty or contaminated suction canister is not mistaken for a sanitized device.

In yet another embodiment of the present invention, a suction canister cover is provided as an accessory for a healthcare product and is designed to mark and indicate the cleanliness status of a suction canister for the view of patients, visitors, and non-medical personnel. More specifically, an indication word, mark or symbol, such as CLEAN or STERILIZED, is clearly visible. The cover product is available in multipacks of, for example, 20, 50, 100 or larger quantities as desired by the user, hospital or manufacturer of the medical devices to be protected with the protective cover.

In a further embodiment of the presently described invention, a protective cover and suction canister combination is described, and includes a suction canister that has a lid and at least one tube in communication with a vacuum source. A protective cover is also provided and formed from a sheet that has opposing sides, and that is formable into an enclosure. The protective cover further comprises a perimeter and a closure element provided within the perimeter of the cover to form the enclosure. The closure element is moveable from a first position to a second position to cover the suction canister, and form the enclosure around the suction canister. The cover has indicia visible on at least one of the opposing sides, the indicia being one of a word or a symbol indicating a condition of the suction canister (e.g., CLEAN or STERILIZED).

In a still further embodiment of the presently described invention, a method of using a protective cover for a medical device is described, and includes the step of initially providing a medical device requiring a sanitary condition and then manufacturing a protective cover. More specifically, the cover is formed from a sheet having opposing sides and that is formable into an enclosure. The cover is further comprised of a perimeter and a closure element provided within the perimeter of the cover to form the enclosure, and the cover has indicia visible on at least one of the opposing sides. The indicia being one of a word or a symbol indicating a condition of the suction canister. Next, the cover is moved from a first position to a second position to cover the medical device and form the enclosure around the medical device. Finally, the cover is removed from the medical device for use in treating a patient.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1a illustrates a side perspective view of one possible embodiment of a medical canister sterilization indicator cap of the present invention in accordance with the disclosed structure;

FIG. 1b illustrates a bottom perspective view of the medical canister sterilization indicator cap of FIG. 1a in accordance with the disclosed structure;

FIG. 2 illustrates a top perspective view of one possible embodiment of a medical canister sterilization indicator cap of the present invention in accordance with the disclosed structure;

FIG. 3 illustrates a bottom perspective view of the underside of one possible embodiment of the medical canister sterilization indicator cap of the present invention in accordance with the disclosed structure, wherein the elasticity of the opening is illustrated;

FIG. 4 illustrates a perspective view of one possible embodiment of the medical canister sterilization indicator cap of the present invention being applied over and substantially covering a clean suction canister in accordance with the disclosed structure; and

FIG. 5 illustrates a perspective view of one possible embodiment of the medical canister sterilization indicator cap of the present invention being removed from a medical canister and properly discarded in accordance with the disclosed structure.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices may be shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a protective covering that easily conforms to and covers medical suction canisters, equipment, medical devices and other components used in the medical field. There is also a long felt need in the art for a quick, accurate and easy method for differentiating between a dirty or used canister or container and a clean, sterilized canister or container. Additionally, there is a long felt need in the art for a canister identification method which requires less effort for the medical professionals to use, and that prevents inadvertent use of a dirty suction canister or equipment for a new patient, thereby preventing cross contamination and the spread of diseases. Finally, there is a long felt need in the art for a sanitized status indicator or identifier for a canister, container, or other medical equipment that is relatively inexpensive to manufacture and safe and easy to use.

The innovative product of the present invention features a disposable sterilization indicator cover for a medical device, such as a suction canister, that is comprised of a top portion forming a cavity that is adapted to receive the medical device or suction canister through an opening at a bottom portion. The indicator cover is further comprised of a channel that includes a rubber band or other elastic defining the opening, wherein the channel extends along the circumference or perimeter of the cover. The exterior surface of the top portion of the cover includes at least one indicator word (e.g., CLEAN, STERILIZED, or the like) or a symbol to visually indicate that the canister is clean and sterilized and ready for use. The indicator word or other indicia allows medical personnel to quickly and decisively determine the cleanliness status of a canister or other medical device for use with patients. The sterilization indicator product of the present invention prevents dirty canisters or devices from being inadvertently used for multiple patients, thereby eliminating the cross contamination that occurs when a dirty canister or other device is used repeatedly in the treatment of multiple patients.

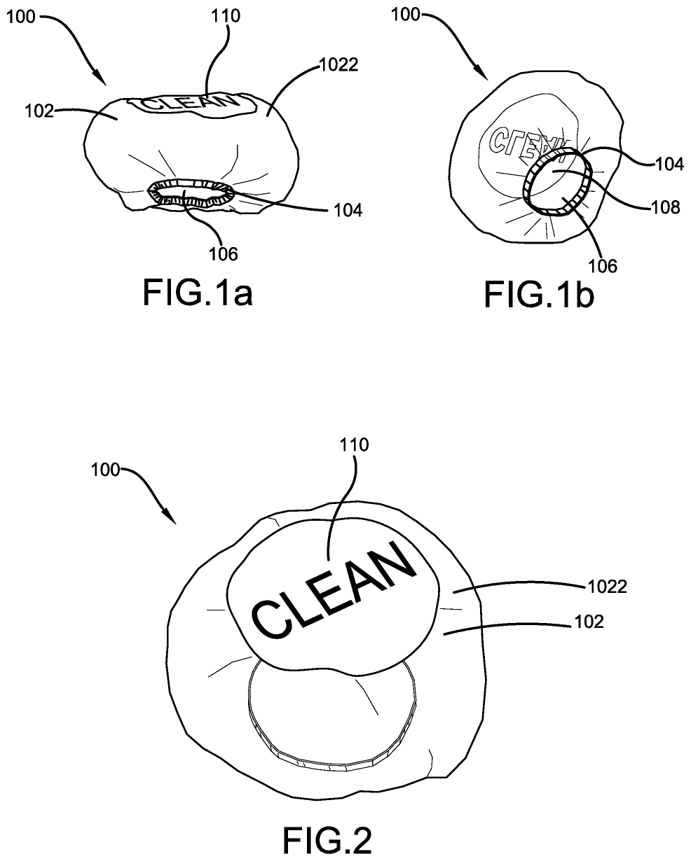

Referring initially to the drawings, FIG. 1a illustrates a side perspective view of one possible embodiment of a medical canister sterilization indicator cap 100 of the present invention in accordance with the disclosed structure, and FIG. 1b illustrates a bottom perspective view of the medical canister sterilization indicator cap 100 of FIG. 1a. Medical canister sterilization indicator cap 100 preferably comprises a top portion 102, an exterior surface 1022 of the top portion 102, and a channel 104, which encloses a rubber band, elastic, draw string or other closure that defines an opening 106, and extends along the circumference or perimeter of the cap 100. The exterior surface 1022 of the top portion 102 has an indication word (e.g., CLEAN or STERILIZED) or symbol 110 printed, imaged or affixed thereto to visually indicate that the medical canister or other device encompassed therein is clean and sterilized, which allows the medical professional to quickly and easily determine whether the canister is appropriate for patient use.

As best shown in FIG. 1b, the cap 100 has a cavity 108 accessible via the opening 106. A user may expand the opening 106 (by stretching the rubber band or elastic) to permit the suction canister, container, medical device to fit into the cavity 108 through the opening 106. In one exemplary embodiment of the present invention, the cap 100 is comprised of a water repellent material or fabric, such as polyurethane or polyurethane laminate, to prevent the canister from becoming prematurely contaminated with any bodily fluids or airborne pathogens. In addition, the material used for the protective cap 100 may also include an anti-microbial coating, or the material itself may be made with the anti-microbial coating embedded in the material at the time of manufacture. The material may also comprise an anti-static coating if the cover is to be used in close proximity to electrical devices that could become damaged by an electrostatic discharge. Other useful coatings for the cap 100 may include an anti-viral coating, an anti-bacterial coating, and/or an UV blocking agent to prevent degradation of the canister or device stored within and being protected by the cap 100.

The channel 104 containing the elastic or rubber band may be formed by one of many different arrangements, such as by sewing, extrusion, adhesion or the like, and generally extends around the inward folded opening perimeter of the cap 100. The elastic band or other closure positioned within the channel 104 is useful for gathering the excess material of the cap 100 after it has been placed over the medical canister or device, and to draw or cinch the cap 100 against the body of the medical device or suction canister, thereby forming a cavity in the top portion 102. In a preferred embodiment of the present invention, the elastic strap or other closure material is preferably comprised of a relatively thin strip of elastic or rubber, typically ¼ inch in width. The closure material may also include a string, a twin, a surgical grade thread, or the like, which may be used in a draw-string like fashion to encompass the canister or device within the cap 100. In some embodiments, the elastic cord may be a nylon bungee cord, sometimes also referred to as “shock cord” or a “stretch cord”.

FIG. 2 illustrates a top perspective view of one possible embodiment of a medical canister sterilization indicator cap 100 of the present invention in accordance with the disclosed structure. More specifically, the sterilization indicator word “CLEAN” 110 is fixed, printed, imaged, stitched or otherwise affixed on the exterior surface 1022 of the cap 100. Alternatively, the word “STERILIZED” may be fixed, printed, imaged, stitched or otherwise affixed on the exterior surface 1022. As previously stated, the words CLEAN or STERILIZED 110 visually indicate that the canister or other medical equipment covered by the cap 100 is clean, sterile and ready for use with a patient. The sterilization indicator word 110 may be provided in any color, such as red, blue, green, neon, etc. In addition, the word or symbol 110 may be printed or imaged in the interior of the cap 100, and visible through the cap or cover if the cap or cover 100 is comprised of a transparent or translucent material. In this way, the printed or imaged word or symbol 110 is not exposed to a contaminated environment, and cannot otherwise be tampered with. The font type and font size of the indicator word 110 may be selected in accordance with the preferences of the user, manufacture, or supplier of the medical device or equipment. Further, any other indicator word 110 may be printed, fixed, stitched or otherwise affixed on the cover 100 based on jurisdictional and/or language requirements.

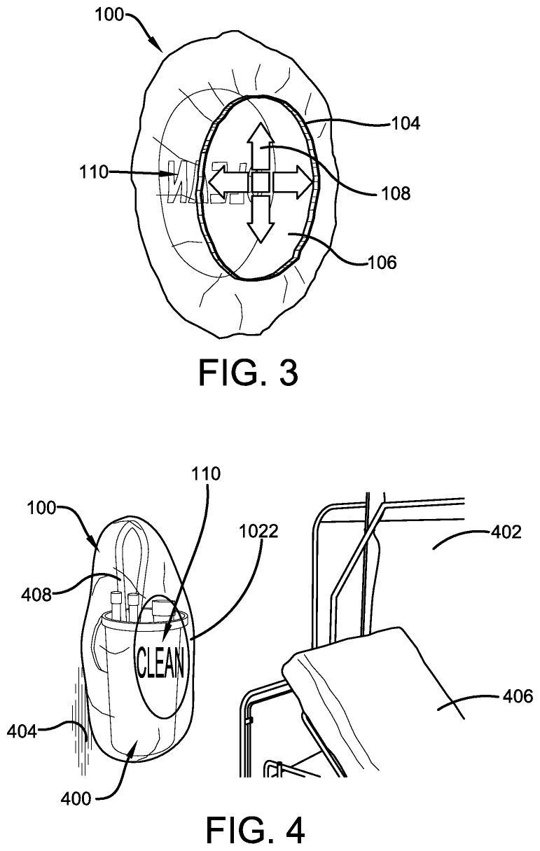

FIG. 3 illustrates a bottom perspective view of the underside of one possible embodiment of the medical canister sterilization indicator cap 100 of the present invention in accordance with the disclosed structure, wherein the elasticity of the opening 106 is illustrated. More specifically, channel 104 is suitable for holding or housing a rubber band, elastic or other closure material that is capable of defining the size and shape of the opening 106, and extends along the circumference or perimeter of the cap 100. The unique design of the cap 100 allows a user to extend the opening 106 by stretching or releasing the draw-string such that the channel 104 increases the size of the opening 106. Further, the opening 106 may conform to the shape of the suction canister or other medical device, such that the top portion 102 covers the canister or medical device in a way that the sterilization indicator word CLEAN 110 is plainly visible to a medical worker on the suction canister or medical device. Additionally, the cap 100 is formed into a bag like structure that envelopes the surface of the medical canister or, if a larger device is to be protected, the bag configuration can be of such a size as to fully encompass the device itself such that there are no portions of the device exposed to the atmosphere.

FIG. 4 illustrates a perspective view of one possible embodiment of the medical canister sterilization indicator cap 100 of the present invention being applied over and substantially covering a clean suction canister 400 in accordance with the disclosed structure. As previously mentioned, suction canister 400 is a temporary storage container for gathering the secretions or fluids removed from the body of a patient during treatment. For example, these fluids or secretions may come from the patient's lungs, stomach, wounds, etc. As shown, the suction canister 400 is attached to a wall 404 of the patient's room 402, or alternatively may be resting on the floor next to the patient's bed 406, or may be provided on a cart which is brought into the area where the treatment is to be performed. The suction canister 400 is like a conventional canister, but has an inlet, an outlet, and related tubing 408. At least one of the tubes 408 is in communication with a vacuum source, and at least one of the other tubes 408 may be applied to the patient. The vacuum source, which may be a wall outlet or a portable compressor, generates a negative pressure within the canister 400 that helps to drain the fluids or secretions from the patient. Examples of tubes 408 that are commonly used with a medical suction canister 400 include, but are not limited to, nasogastric, respiratory suction catheters, G-tubes, and wound drains.

Prior to its use, the canister 400 is sterilized and covered with the medical canister sterilization indicator cap 100 of the present invention, indicating its availability for patient use. The medical canister sterilization indicator cap 100 is preferably comprised of a transparent or translucent plastic material so that the user can easily see that no secretions or fluids are present in the canister 400. The cap 100 covers all of the components of the canister 400 that may require sterilization, such as the tubes 408, to keep the suction canister 400 and its various components contamination free and ready for use by a patient. A sterilization indicator word (e.g., CLEAN) or other symbol 110 is written, printed or imaged on the interior or exterior surface 1022 of the top surface 102 of the cap 100 so that the same is clearly visible to the medical personnel. For example, if the word or symbol 110 is printed on the interior surface of the cap 100, it remains visible through the transparent or translucent material of the cover 100.

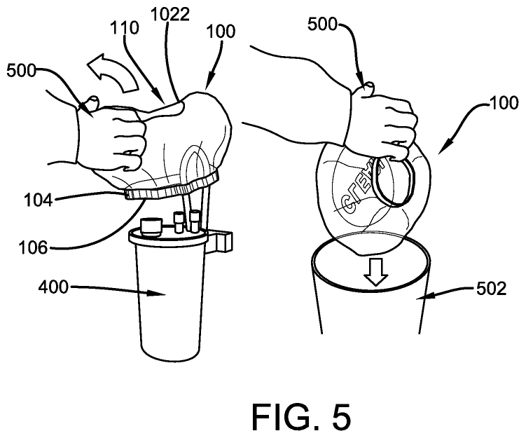

FIG. 5 illustrates a perspective view of one possible embodiment of the medical canister sterilization indicator cap 100 of the present invention being removed from a medical canister 400 and properly discarded in accordance with the disclosed structure. Alternatively, the cap 100 may be appropriately recycled after removing the same from the canister 400 or other medical device. More specifically, to use a clean and sterilized canister 400, a user 500 removes the cap 100 from the canister 400 by stretching the channel 104 via the elastic closure or drawstring, thereby increasing the size of the opening 106 of the cap 100 to allow the canister 400 or other medical device to pass therethrough. After removal of the cap or cover 100, the user 500 discards the cap 100 in a safe manner in, for example, a dustbin 502, recycling container, or other medical waste receptacle.

The cap 100 itself is preferably generally circular or oval in shape, and the opening 106 measures between approximately ten and sixteen inches in diameter, although larger or smaller diameters may also be used in order to accommodate different sizes and shapes of the medical devices or equipment to be enclosed therein. For example, the cap 100 may cover suction canisters 400 ranging in size from approximately 600 to 1600 cc, and of various shapes, though such capacities are not meant to be a limitation on the present invention. The cap 100 of the present invention may also be manufactured in a transparent or translucent color, or in a variety of different colors, patterns, or decorative prints to suit user need or preference.

In one embodiment, a method of constructing the canister cap 100 of the present invention involves first cutting a circular piece of transparent or translucent plastic or glassine material of a desired perimeter or shape. A strip of closure material is then cut and preferably has a length that is less than the perimeter of the transparent or translucent elastic material. The strip of closure material is then placed sufficiently inward of the outer perimeter of the shaped piece of transparent or translucent material so as to leave enough material to fold over and cover the strip entirely and to be sewn to the material. In this manner, the majority of the strip of closure material is generally encapsulated in the channel formed by the folded over material.

The disposable medical device cover 100 of the present invention exhibits an integrally molded, seamless, unitary one-piece construction, and can be manufactured from, for example, a latex based material, a polyurethane compound, or any other suitable material, such as glassine. The selected material is preferably flexible, elastic (i.e., stretchable), and thus is easily adaptable or conformable to the shape of most any size suction canister 400, or other medical device needing a protective cover. Thus, once fitted, the protective cover 100 fits snugly over the suction canister 400 or other medical device. The selected material from which the cover 100 is fashioned is also preferably transparent or translucent. Thus, once fitted onto the suction canister or other medical device, the transparent or translucent nature of the inventive protective cover 100 facilitates unimpeded view of the canister 400 stored therein. As previously mentioned, the material from which the disposable cover 100 is manufactured may be also be impregnated with an anti-bacterial compound to protect the canister 400 and associated tubing 408 from dirt, bacterial, germs, microbes, viruses and the like.

Several of the characteristics of the latex based material or polyurethane compound (e.g., Grade SR-9000RT, plus the anti-bacterial compound) from which the disposable canister cap 100 is fashioned are transparent, stretchable, and exhibit sufficient tensile strength and tackiness. The transparency feature refers to the clarity of the material and, in the present invention, the disposable canister cap 100 is clear, not cloudy or milky colored, that is, without color distortion. The tensile strength refers to the anti-tearing or shearing of the latex based material or polyurethane compound comprising the disposable canister cap 100, which is highly resistant to tearing and/or puncturing. More specifically, the tensile strength of the disposable cover 100 has a rating or coefficient of “700”, which means that the disposable cover 100 will stretch up to seven times its original dimensions (i.e., in a relaxed state), before the material fails. This characteristic maximizes the flexibility, elasticity and stretchability of the disposable cover 100 when used as intended. Finally, the material comprising the disposable cover 100, including the anti-bacterial compound, exhibits a minimum of tackiness or stickiness to the touch for providing a non-sticky surface.

To maintain a safe, sterile medical canister 400, the indicator cap 100 is a surefire way of safeguarding sanitized suction canisters from mixing with those that have been previously used or are otherwise contaminated with infectious bacteria, virus, germs, microbes and the like. The indicator cap 100 may be comprised from sterile, medical grade plastic, and features an elastic closure that can be mounted on any suction canister 400. With the aid of rubber bands, elastic bands, or other closures, the canister cover 100 stays securely in place until its removal is required. The disposable covers 100 are also preferably latex free to avoid potential allergic reactions on the part of both staff and patient. The cap 100 may further comprise one or more protective coatings to protect the cover 100 and the medical devices 400 stored therein.

From the foregoing, it will be appreciated that the indicator cap 100 of the present invention provides numerous advantages. For example, the indicator cap 100 of the present invention is cost-effective, easy and safe to use for purposes of indicating a cleaned and sterilized suction canister 400 from one that is contaminated or previously used without being re-sanitized. The cap 100 is also attractive in appearance and adjustable in size to easily cover or substantially encompass canisters of various sizes, as well as other medical devices. Other advantages include the speed of covering and removal of the indicator cap 100, greater variety in available coloring patterns that may be developed, and convenient disposal or recycling after a single use.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “medical canister sterilization indicator cap”, “canister cover”, “cap”, “cover”, “plastic cover”, “inventive cover” and “disposable cap” are interchangeable and refer to the medical canister sterilization indicator cap 100 of the present invention. As used herein “canisters”, “suction canisters” and “medical containers” are used interchangeably.

Notwithstanding the forgoing, the medical canister sterilization indicator cap 100 of the present invention can be of any suitable size and configuration as is known in the art without affecting the overall concept of the invention, provided that it accomplishes the above stated objectives. One of ordinary skill in the art will appreciate that the size, configuration and material of the medical canister sterilization indicator cap 100 as shown in the FIGS. are for illustrative purposes only, and that many other sizes of the medical canister sterilization indicator cap 100 are well within the scope of the present disclosure. Although the dimensions of the medical canister sterilization indicator cap 100 are important design parameters for user convenience, the medical canister sterilization indicator cap 100 may be of any size that ensures optimal performance during use and/or that suits user need and/or preference. It is further contemplated that a single medical canister sterilization indicator cap 100 may be both circular and oval shaped, and of varying dimensions, thereby allowing the user to choose which configuration and size to choose for a particular application. The cover of the present invention may be of any particular shape, square or rectangular for example to cover a monitor, screen, tray or similarly shaped medical device. Any geometric shape is possible depending on the device require the protection and identification of status. The possibilities are endless.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A protective cover for medical equipment, comprising;
a sheet of material, the sheet of material having opposing sides and formable into an enclosure, wherein the sheet of material further comprises a perimeter and a closure element provided within the perimeter of the sheet of material to form the enclosure;
the closure element moveable from a first position to a second position to cover the medical equipment and to form the enclosure around the medical equipment; and
the sheet of material having an indicia visible on at least one of the opposing sides, the indicia being one of a word or a symbol to indicate a condition of the medical equipment.

2. The protective cover for medical equipment of claim 1, wherein the medical equipment is selected from a group including a suction canister, a monitor and a surgical tray.

3. The protective cover for medical equipment of claim 2, wherein the suction canister comprises at least one tube and a lid.

4. The protective cover for medical equipment of claim 1, wherein the sheet of material is selected from a group including a plastic, a polyurethane and a glassine.

5. The protective cover for medical equipment of claim 4, wherein the sheet or material is transparent or translucent.

6. The protective cover for medical equipment of claim 1, wherein the sheet of material is provided with at least one of an anti-microbial, an anti-viral or an anti-bacterial solution.

7. The protective cover for medical equipment of claim 1, wherein the sheet of material is provided with at least one of an anti-static or an UV blocking formulation.

8. The protective cover for medical equipment of claim 1, wherein the sheet of material has a coefficient of at least 700.

9. The protective cover for medical equipment of claim 1, wherein the indicia is provided by at least one of a printing, an imaging, a stitching, or a sewing.

10. The protective cover for medical equipment of claim 1, wherein the sheet of material is a single use material that is recyclable.

11. A protective cover and suction canister combination comprising;
a suction canister having a lid and at least one tube in communication with a vacuum source;
a protective cover formed from a sheet having opposing sides and formable into an enclosure, wherein the protective cover further comprises a perimeter and a closure element provided within the perimeter of the protective cover to form the enclosure;
the closure element moveable from a first position to a second position to cover the suction canister and form the enclosure around the suction canister; and
the protective cover having an indicia visible on at least one of the opposing sides, the indicia being one of a word or a symbol indicating a condition of the suction canister.

12. The protective cover and suction canister combination of claim 11, wherein the protective cover is selected from a material group including a plastic, a polyurethane and a glassine.

13. The protective cover and suction canister combination of claim 11, wherein the protective cover is transparent or translucent.

14. The protective cover and suction canister combination of claim 11, wherein the protective cover is provided with at least one of an anti-microbial, an anti-viral or an anti-bacterial solution.

15. The protective cover and suction canister combination of claim 11, wherein the protective cover is provided with at least one of an anti-static or an UV blocking formulation.

16. The protective cover and suction canister combination of claim 11, wherein the indicia is one of a word or a symbol indicating a sterilized status of the suction canister.

17. The protective cover and suction canister combination of claim 11, wherein the indicia is provided by at least one of a printing, an imaging, a stitching, or a sewing.

18. A method of using a protective cover for a medical device, the method comprising the steps of;
providing a medical device requiring a sanitary condition;
manufacturing a protective cover, the protective cover formed from a sheet having opposing sides and formable into an enclosure, the protective cover further having a perimeter and a closure element provided within the perimeter of the protective cover to form the enclosure and the protective cover having an indicia visible on at least one of the opposing sides, wherein the indicia is one of a word or a symbol indicating a condition of the medical device;
moving the protective cover from a first position to a second position to cover the medical device and form the enclosure around the medical device; and
removing the cover from the medical device for use in treating a patient.

19. The method of using a protective cover for a medical device of claim 18, wherein the protective cover is provided with at least one of an anti-microbial, an anti-viral, and an anti-bacterial solution, and further wherein the protective cover is provided with at least one of an anti-static formulation and an UV blocking formulation.

20. The method of using a protective cover for a medical device of claim 18, wherein the protective cover is selected from a material group including a plastic, a polyurethane and a glassine.
